(19) United States
(12) Patent Application Publication
(10) Pub. No.: US 2009/0124675 A1
(43) Pub. Date: May 14, 2009

(54) INHIBITORS OF ANTI-APOPTOTIC PROTEINS

(75) Inventor: Maurizio Pellecchia, La Jolla, CA (US)

Correspondence Address: DLA PIPER LLP (US), 4365 EXECUTIVE DRIVE, SUITE 1100, SAN DIEGO, CA, 92121-2133, US

(73) Assignee: Burnham Institute for Medical Research, La Jolla, CA (US)

(21) Appl. No.: 12/253,921

(22) Filed: Oct. 17, 2008

Related U.S. Application Data
(60) Provisional application No. 60/981,466, filed on Oct. 19, 2007.
(60) Provisional application No. 61/057,129, filed on May 29, 2008.

(51) Int. Cl. A61K 31/426; C07D 277/36; A61P 35/04; C12Q 1/00; C12N 5/02

(52) U.S. Cl. 514/369; 548/183; 435/4; 435/375

(57) ABSTRACT

Various compounds comprising a thiazolidine ring are described as well as the use of such compounds to inhibit at least one BCL-2 protein family member. One of the compounds described has the structure the structure A,

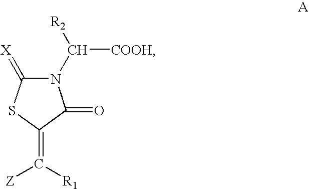

wherein each of R₁, and R₂ comprises hydrogen, a substituted or unsubstituted straight-chained aliphatic group, a halogen, an alkoxyl, a halogen-substituted alkyl, a halogen-substituted alkoxyl, hydroxyl, carboxyl, cyano group, an amido group, a substituted or unsubstituted cycloaliphatic group, a substituted or unsubstituted aromatic group, or a substituted or unsubstituted heterocyclic group; X comprises oxygen, sulfur, or imino group; and Z comprises a moiety such as naphthaline or dehydronaphthaline, among others.

### CROSS-REFERENCE TO RELATED APPLICATIONS

This application claims priority under 35 U.S.C. §119(e) to each of U.S. Patent Application Ser. No. 60/981,466 filed Oct. 19, 2007 and U.S. Patent Application Ser. No. 61/057,129 filed May 29, 2008, each of which is herein incorporated by reference in its entirety.

### BACKGROUND

1. Field of the Invention

The present invention relates generally to compounds used for treating a variety of disorders, diseases and pathologic conditions, and more specifically, to the use of chemical compounds comprising thiazolidine moiety, to treat such disorders.

2. Background Information

The apoptotic cascade in cells is known to lead to cell death. When anti-apoptotic proteins, such as BCL-2 family proteins, are overproduced by the cells, uncontrollable cell growth may ensue, potentially leading to the development of various serious diseases, disorders, and pathologies, particularly cancer.

Therefore, a need exists to inhibit anti-apoptotic proteins, such as the BCL-2 family proteins. Various potential BCL-2 antagonists have been previously identified. However, none of these compounds inhibits all six proteins in the BCL-2 family, i.e., all of the following proteins: BCL-X_{L}, BCL-2, BCL-W, BCL-B, BFL-1, and MCL-1. For example, none of the previously identified synthetic BCL-2 antagonists was effective at inhibiting the protein BFL-1. Therefore, the efficiency of such antagonists is not as high as desired. In addition, the existing antagonists are characterized by other drawbacks, such as insufficiency or safety issues.

In view of the above drawbacks and deficiencies of existing BCL-2 inhibitors, new antagonists of anti-apoptotic proteins, such as BCL-2 family proteins, are desired. It is desirable that such new antagonists be safer and more effective than the existing compounds.

### SUMMARY

According to one embodiment of the invention, there are provided compounds having the structure A, or a pharmaceutically acceptable salts, hydrates, N-oxides, or solvates thereof:

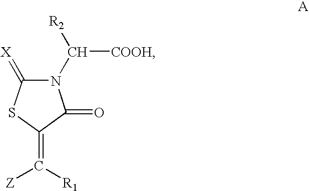

In the compounds having the structure A, each of R₁ and R₂ may be hydrogen, a substituted or unsubstituted straight-chained aliphatic group, a halogen, an alkoxyl, a halogen-substituted alkyl, a halogen-substituted alkoxyl, hydroxyl, carboxyl, cyano group, an amido group, a substituted or unsubstituted cycloaliphatic group, a substituted or unsubstituted aromatic group, or a substituted or unsubstituted heterocyclic group; X may be oxygen, sulfur, or imino group; and Z is a moiety that may have any of the structures I, II, and III:

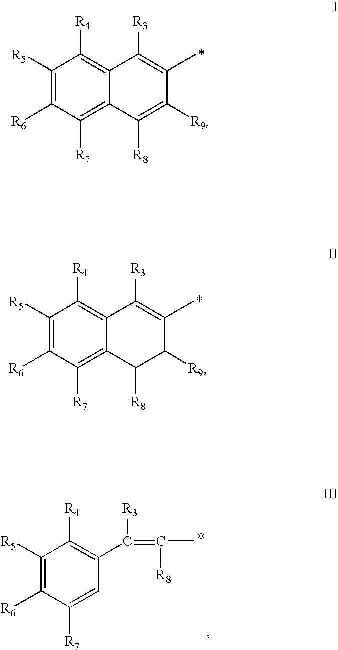

In each of the moieties I, II, and III, each of R₃, R₄, R₅, R₆, R₇, R₈, and R₉ may be hydrogen, a substituted or unsubstituted straight-chained aliphatic group, a halogen, an alkoxyl, a halogen-substituted alkyl, a halogen-substituted alkoxyl, hydroxyl, carboxyl, cyano group, an amido group, a substituted or unsubstituted cycloaliphatic group, a substituted or unsubstituted aromatic group, or a substituted or unsubstituted heterocyclic group. In each of the moieties I, II, and III, the *symbol indicates the point of attachment of the moiety Z of the immediately adjacent carbon in the C(═)—R₁ structure.

According to another embodiment of the present invention, a method for treating cancer or autoimmune diseases is provided, comprising administering to a subject in need thereof a therapeutically effective amount of at least one compound having the structure 11, or a pharmaceutically acceptable salt, hydrate, N-oxide, or solvate thereof:

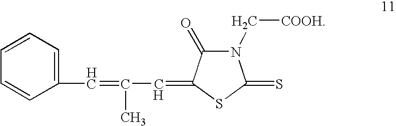

### BRIEF DESCRIPTION OF FIGURES

FIG. 1 demonstrates a predicted binding mode of a compound of the invention to a protein of the BCL-2 family.

FIG. 2A demonstrates a predicted binding mode of another compound of the invention to a protein of the BCL-2 family.

FIG. 2B demonstrates a predicted binding mode of yet another compound of the invention to a protein of the BCL-2 family.

FIG. 3 provides NMR data showing binding of a compound of the invention to a protein of the BCL-2 family.

FIGS. 4 and 5 provide NMR data showing binding of other compounds of the invention to a protein of the BCL-2 family.

FIGS. 6, 7, and 8 are the reaction schemes demonstrating schematically some exemplary ways of making some of the compounds of the invention.

### DETAILED DESCRIPTION

The following terms, definitions and abbreviations apply:

The general terms “alkyl,” and “alkoxy refer to both straight-chain and branched groups; references to individual radicals include specifically either straight-chain or branched groups, but not both. For instance, a reference to “propyl” includes only the straight-chain radical while a reference to “isopropyl” includes only the branched group.

The term “alkyl” refers to a monovalent straight or branched chain hydrocarbon group. Examples of alkyl structures that can be used include (C₁-C₆)alkyls such as be methyl, ethyl, propyl, isopropyl, butyl, isobutyl, sec-butyl, tert-butyl, pentyl, 3-pentyl, or hexyl.

The term “halo” refers herein to fluoro, chloro, bromo, or iodo. The term “haloalkyl” refers to a halogen-substituted alkyl, such as halo(C₁-C₆)alkyl, for example, iodomethyl, bromomethyl, chloromethyl, fluoromethyl, trifluoromethyl, 2-chloroethyl, 2-fluoroethyl, 2,2,2-trifluoroethyl, or pentafluoroethyl.

The terms “alkoxyl” or “alkoxy” refer to the moiety —O-alkyl, wherein alkyl is as defined above. Examples of alkoxy structures that can be used include (C₁-C₆)alkoxy radicals, such as methoxy, ethoxy, propoxy, isopropoxy, butoxy, iso-butoxy, sec-butoxy, tert-butoxy, pentoxy, 3-pentoxy, or hexyloxy.

The term “carboxyl or “carboxy” refer to the moiety combining carbonyl and hydroxyl functional groups and having the structure —C(═O)—OH.

The term “aliphatic” refers to a moiety containing solely straight or branched chain arrangements of carbon atoms and lacking any rings or aromaticity.

The term “cycloaliphatic” refers to any ring structure other than an aromatic structure.

The term “aromatic” refers to a cyclically conjugated molecular entity with stability, due to delocalization, significantly greater than that of a hypothetical localized structure, such as the Kekulé structure.

The term “aryl” refers to a phenyl radical or an ortho-fused bicyclic carbocyclic structure having at least one aromatic ring. Some examples of aryls include, but are not limited to, phenyl, indenyl, or naphthyl, biphenyl, dihydronaphthyl, tetrahydronaphthyl, indanyl, azulenyl, anthryl, phenanthryl, fluorenyl, and pyrenyl.

The terms “heterocycle” or “heterocycle” refer to a monovalent saturated or unsaturated group having a single ring or multiple condensed rings, from 1 to 8 Carbon atoms and from 1 to 4 hetero atoms selected from nitrogen, sulfur or oxygen within the ring. The heterocylic groups may be optionally substituted with 1 to 3 substituents, such as, but not limited to, a C₁₋₁₀alkyl, a C₁₋₁₀alkoxyl, an aryl, halogen, —OH, —SH, —CN, —NO₂, or trihalomethyl. Some examples of heterocyclic groups include, but are not limited to, thienyl, furyl, pyranyl, pyrrolyl, indolyl, benzimidazolyl, pyridyl, etc.

The term “cyano” refers to the functional group, —CN, i.e., the group where a carbon and a nitrogen atoms are joined with a triple bond.

The terms “imine” or imino refers to the functional group —CR═N— where a carbon and a nitrogen atoms are joined with a doble bond.

The terms “amide” or “amido” refer to a moiety CON(R₁R₂), where each of R₁ and R₂ is independently hydrogen or an alkyl.

The term “thiazolidine” refers to a compound containing a moiety derived from the compound having the formula:

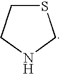

The term “naphthalene” refers to a compound containing a moiety derived from the compound having the formula:

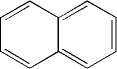

The term “dihydronaphthalene” refers to a compound containing a moiety derived from a partially hydrogenated naphthalene, for example, derived from the compound having the formula:

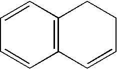

The term “patient” refers to organisms to be treated by the methods of the present invention. Such organisms include, but are not limited to, humans. In the context of the invention, the term “subject” generally refers to an individual who will receive or who has received treatment described below (e.g., administration of the compounds of the invention, and optionally one or more additional therapeutic agents).

The term “BCL-2 family of proteins” refers to the family of proteins that currently includes at least the following proteins: BCL-X_{L}, BCL-2, BCL-W, BCL-B, BFL-1, and MCL-1.

According to one embodiment of the invention, compounds having the structure A, or pharmaceutically acceptable salts, hydrates, or solvates thereof, are provided for treatment of various diseases, disorders, and pathologies:

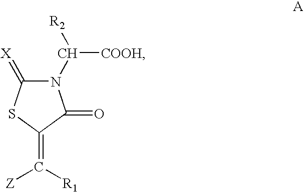

In the compounds having the structure A, each of R₁ and R₂ comprises hydrogen, a substituted or unsubstituted straight-chained aliphatic group, a halogen, an alkoxyl, a halogen-substituted alkyl, a halogen-substituted alkoxyl, hydroxyl, carboxyl, cyano group, an amido group, a substituted or unsubstituted cycloaliphatic group, a substituted or unsubstituted aromatic group, or a substituted or unsubstituted heterocyclic group; X comprises oxygen, sulfur, or imino group; and Z is a moiety selected from the group having the structures I, II, and III:

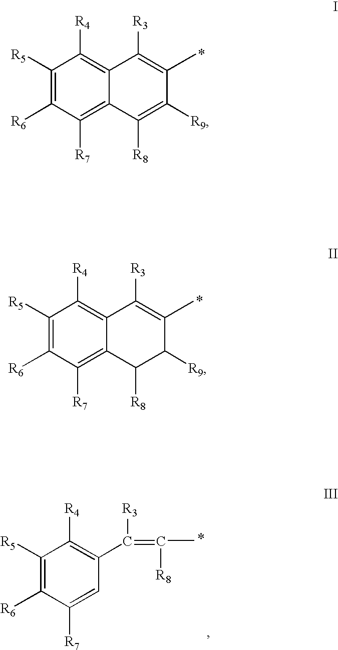

In each of the moieties I, II, and III, each of R₃, R₄, R₅, R₆, R₇, R₈, and R₉ may be any of hydrogen, a substituted or unsubstituted straight-chained aliphatic group, a halogen, an alkoxyl, a halogen-substituted alkyl a halogen-substituted alkoxyl, hydroxyl, carboxyl, cyano group, an amido group, a substituted or unsubstituted cycloaliphatic group, a substituted or unsubstituted aromatic group, or a substituted or unsubstituted heterocyclic group. In each of the moieties I, II, and III, the *symbol indicates the point of attachment of the moiety Z of the immediately adjacent carbon in the C(═O)—R₁, structure.

Accordingly, in the embodiments, where in the compound having the structure A, Z is a substituted naphthalene group (i.e., moiety I), the compound having such a substitutent Z may have the structure AI:

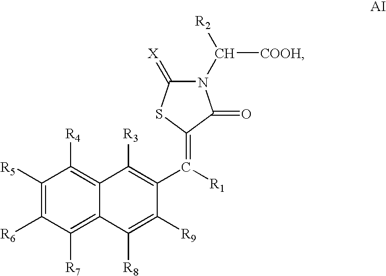

In the compound having the structure AI, each of R₁, R₂, R₃, R₄, R₅, R₆, R₇, R₈, and R₉ may be, independently, hydrogen, a substituted or unsubstituted straight-chained aliphatic group, a halogen, an alkoxyl, a halogen-substituted alkyl, a halogen-substituted alkoxyl, hydroxyl, carboxyl, cyano group, an amido group, a substituted or unsubstituted cycloaliphatic group, a substituted or unsubstituted aromatic group, or a substituted or unsubstituted heterocyclic group. For example, each of R₁, R₂, R₃, R₄, R₅, R₆, R₇, R₈, and R₉ may be independently any of hydrogen, methyl, n-propyl, iso-propyl, fluorine, chlorine, bromine, phenyl, hydroxyl, methoxy, trifluoromethyl, trifluoromethoxy, cyano, carboxyl, or —C(O)NH₂.

In other embodiments, in the compound having the structure A, Z may be a substituted dihydronaphthalene group (i.e., moiety II), and the compound having such a substitutent Z may have the structure AII:

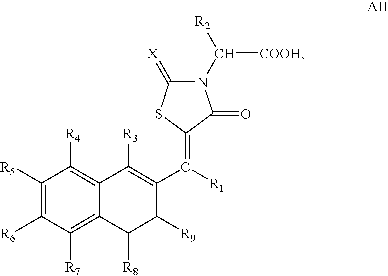

In the compound having the structure AII, each of R₁, R₂, R₃, R₄, R₅, R₆, R₇, R₈, and R₉ may be, independently, hydrogen, a substituted or unsubstituted straight-chained aliphatic group, a halogen, an alkoxyl, a halogen-substituted alkyl, a halogen-substituted alkoxyl, hydroxyl, carboxyl, cyano group, an amido group, a substituted or unsubstituted cycloaliphatic group, a substituted or unsubstituted aromatic group, or a substituted or unsubstituted heterocyclic group. For example, each of R₁, R₂, R₃, R₄, R₅, R₆, R₇, R₈, and R₉ may be, independently, hydrogen, methyl, n-propyl, iso-propyl, fluorine, chlorine, bromine, phenyl, hydroxyl, methoxy, trifluoromethyl, trifluoromethoxy, cyano, carboxyl, or —C(O)NH₂.

In other embodiments, in the compound having the structure A, Z may be moiety III, and the compound having such a substitutent Z may have the structure AIII:

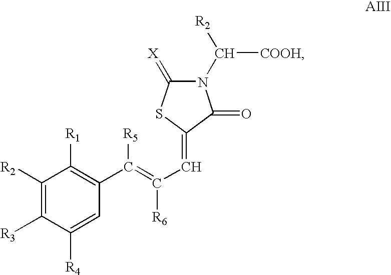

In the compound having the structure AIII, each of R₁, R₂, R₃, R₄, R₅, R₆, and R₇ may be, independently, hydrogen, a substituted or unsubstituted straight-chained aliphatic group, a halogen, an alkoxyl, a halogen-substituted alkyl, a halogen-substituted alkoxyl, hydroxyl, carboxyl, cyano group, an amido group, a substituted or unsubstituted cycloaliphatic group, a substituted or unsubstituted aromatic group, or a substituted or unsubstituted heterocyclic group. For example, each of R₁, R₂, R₃, R₄, R₅, R₆, and R₇ may be, independently, hydrogen, methyl, n-propyl, iso-propyl, fluorine, chlorine, bromine, phenyl, hydroxyl, methoxy, trifluoromethyl, trifluoromethoxy, cyano, carboxyl, or —C(O)NH₂.

Some exemplary compounds described by structure A that can be used include 2-[5-(2-methyl-3-phenylallylidene)-4-oxo-2-thioxothiazolidin-3-yl]-2-phenylacetic acid (compound 1) and 3-methyl-2-[5-(2-methyl-3-phenylallylidene)-4-oxo-2-thioxothiazolidin-3-yl]butanoic acid (compound 2) shown below:

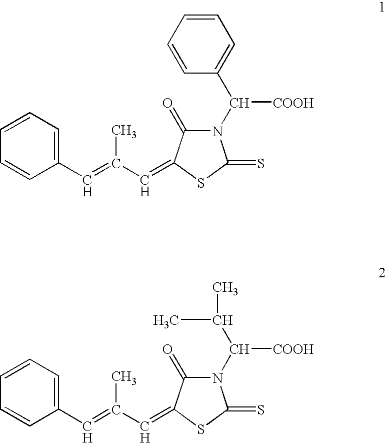

As can be seen, exemplary compounds 1 and 2 are within the purview of the structure AIII, wherein, each of R₁, R₂, R₃, R₄, and R₅ is hydrogen, R₆, is methyl, X is sulfur, and R₇ is phenyl (compound 1) or iso-propyl (compound 2).

Some additional exemplary compounds described by structure A that can be used include the following compounds:
2-[5-(1-isopropyl-2,3-dimethoxy-7-methylnaphthalen-6-yl-methylene)-4-oxo-2-thioxothiazolidin-3-yl]-3-methylbutanoic acid (compound 3);
2-[5-((2,3-dihydroxy-1-isopropyl-7-methylnaphthalen-6-yl)methylene)-4-oxo-2-thioxothazolidin-3-yl]-3-methylbutanoic acid (compound 4);
2-[5-((4-bromo-1,2-dihydro-6,7-dimethoxynaphthalen-3-yl)methylene)-4-oxo-2-thioxothiazolidin-3-yl]-3-methylbutanoic acid (compound 5);
2-[5-((4-bromo-1,2-dihydro-6,7-dihydroxynaphthalen-3-yl)methylene)-4-oxo-2-thioxothiazolidin-3-yl]-3-methylbutanoic acid (compound 6);
2-[5-((1,2-dimethoxynaphthalen-3-yl)methylene)-4-oxo-2-thioxothiazolidin-3-yl]-3-methylbutanoic acid (compound 7);
2-[5-((naphthalen-2-yl)methylene)-4-oxo-2-thioxothiazolidin-3-yl]acetic acid (compound 8);
3-methyl-2-[4-oxo-5-(3-phenylallylidene)-2-thioxothiazolidin-3-yl]butanoic acid (compound 9); and
2-[4-oxo-5-(3-phenylallylidene)-2-thioxothiazolidin-3-yl]propanoic acid (compound 10).

The structures of the above-identified compounds 3 through 10 are shown below:

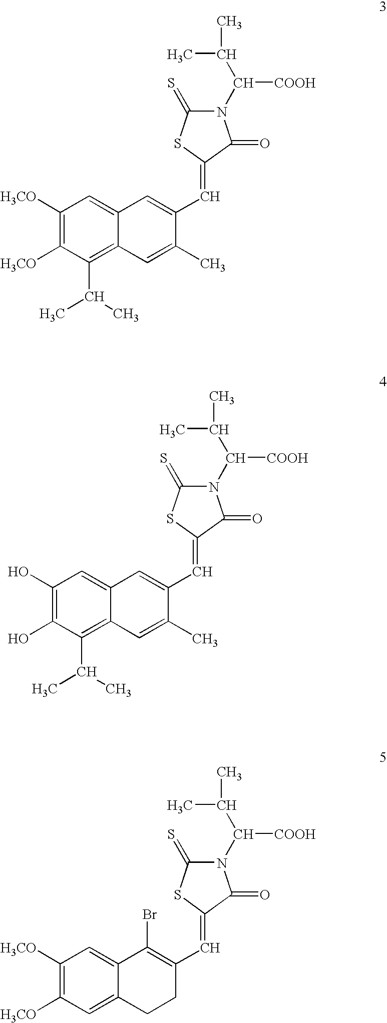

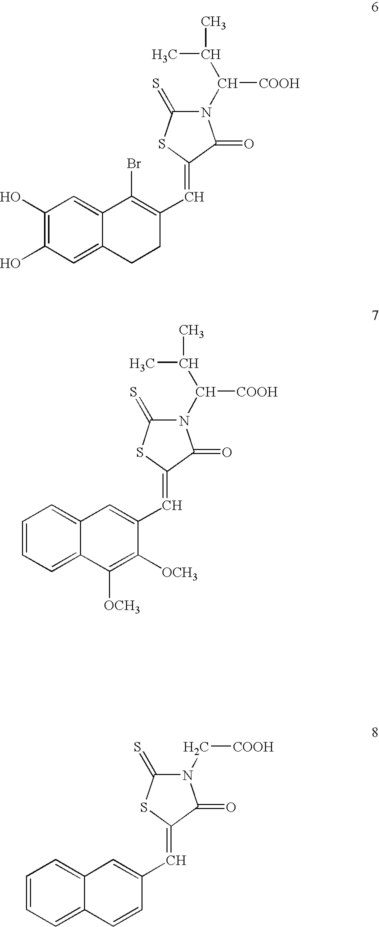

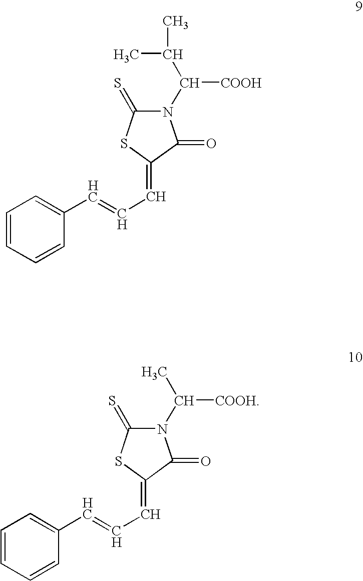

Some compounds of the invention may have a chiral center and can be isolated in optically active and racemic forms. Some compounds may exhibit polymorphism. The compounds of the present invention include any racemic, optically-active, polymorphic, or stereoisomeric forms or mixtures thereof, which possess the useful properties described herein. If desired, optically active forms can be prepared using commonly known techniques, e.g., by resolution of the racemic form by recrystallization techniques, by synthesis from optically-active starting materials, by chiral synthesis, or by chromatographic separation using a chiral stationary phase.

In one embodiment, a method is provided for inhibition of an anti-apoptotic family of proteins BCL-2. The method includes contacting a BCL-2 protein with at least one of above-identified compounds under conditions that are favorable for contacting a BCL-2 protein and a compound of the invention. While not wanting to be bound to a particular mechanism, the above-identified compounds of the present invention are believed to be capable of inhibiting six proteins of the BCL-2 family, e.g., are capable of inhibiting all of such proteins as BCL-X_{L}, BCL-2, BCL-W, BCL-B, BFL-1, and MCL-1.

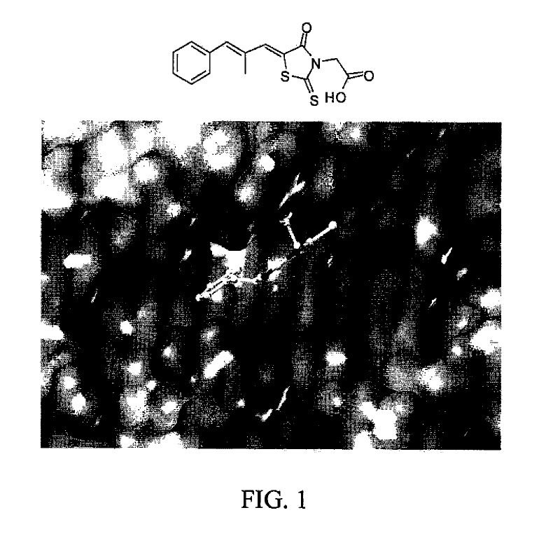

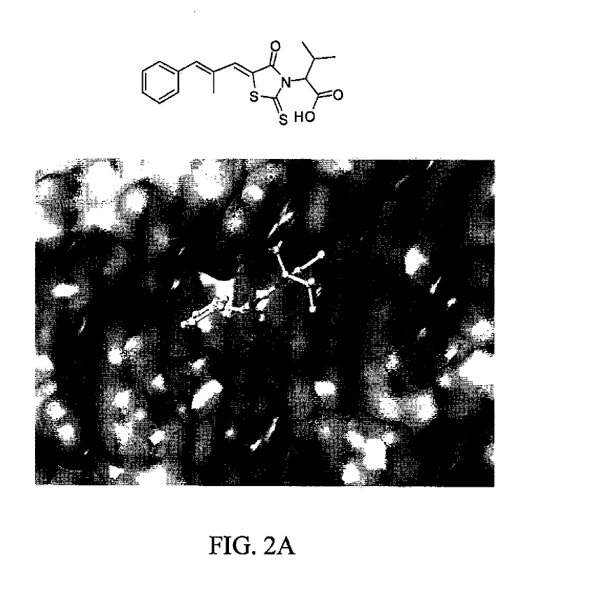

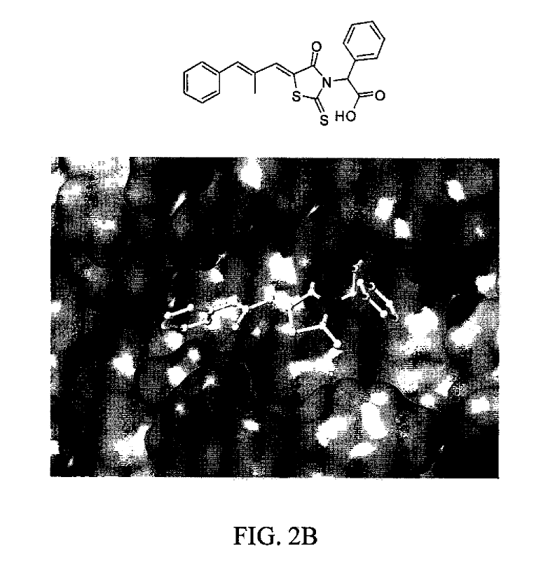

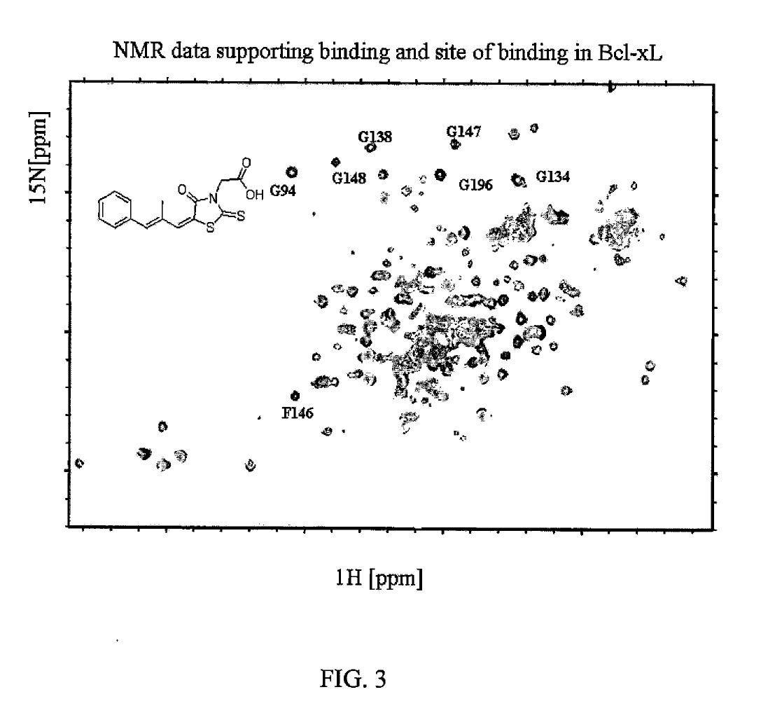

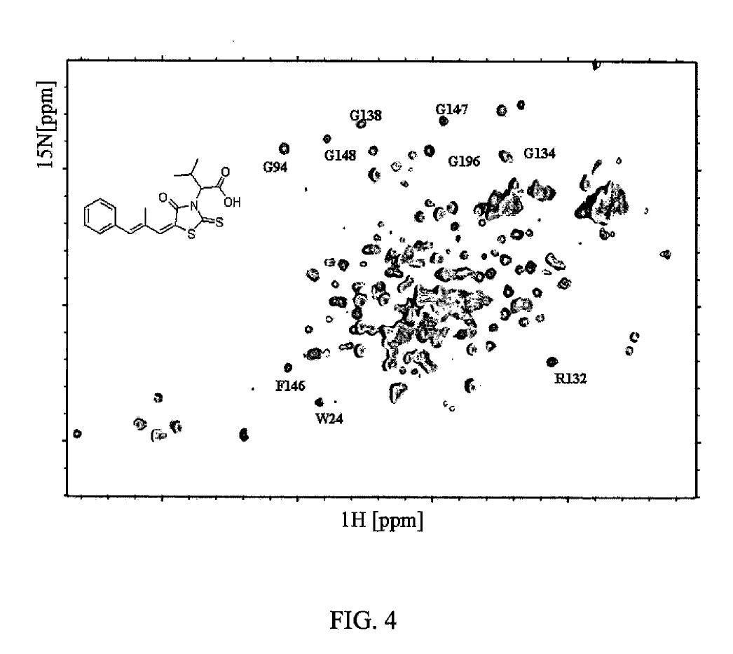

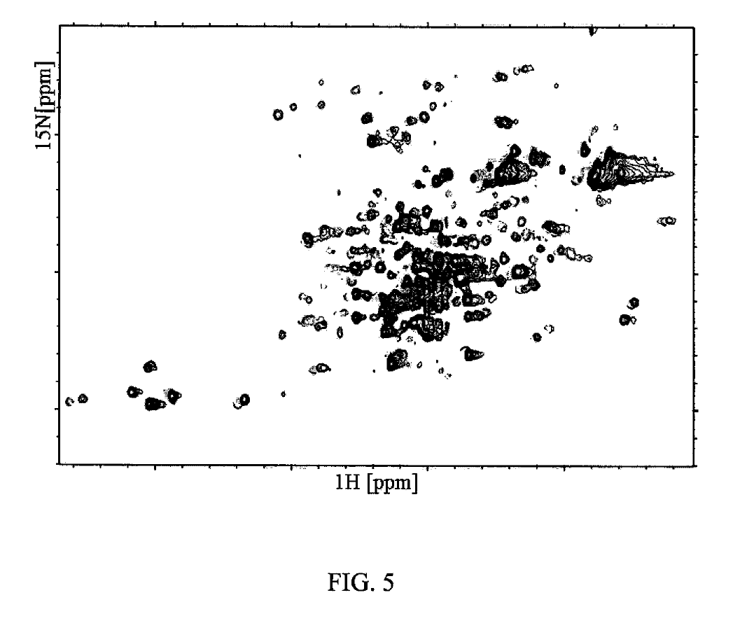

Predicted binding mode of some compounds of the present invention to a BCL-2 protein is illustrated by FIG. 1 (binding of compound 11 shown below), FIG. 2A (binding of compound 1 shown above), and FIG. 2B (binding of compound 2 shown above). Binding is further illustrated by FIGS. 3-5 showing NMR data that support the conclusion that binding had occurred and indicate the site of binding in BCL-X_{L} protein. FIG. 3 shows such NMR data for compound 11 shown below in comparison with compositions having no inhibitor. FIG. 4 and FIG. 5 show such NMR data for compounds 6 and 7 shown above. The inhibition was also evaluated by measuring dissociation constant (K_{d}) values for some compounds of the invention. Such inhibition data are shown in Table 1.

TABLE 1
Inhibition Data for Some Compounds of the Invention

| | Compound | | | | |
|---|---|---|---|---|---|
| | 1 | 2 | 9 | 10 | 11 |
| K_{d}, μM | 1.0 | 5.7 | 10.6 | 34.4 | 11.8 |

According to other embodiments, a method is provided for treating a disease or disorder. The method can include administering to a subject in need of such treatment, an effective amount of any above-described compound, or pharmaceutically acceptable salts, hydrates, or solvates thereof. Non-limiting examples of the diseases or disorders that can be treated are cancer and autoimmune diseases.

According to another embodiment, a method is provided for treating cancer. The method comprises administering to a subject in need thereof a therapeutically effective amount of 2-[5-(2-methyl-3-phenylallylidene)-4-oxo-2-thioxothiazolidin-3-yl]acetic acid, which is the compound having the structure 11, or a pharmaceutically acceptable salt, hydrate, N-oxide, or solvate thereof:

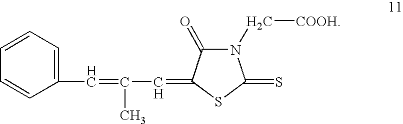

As can be seen, compound 11 is within the purview of the structure AIII, wherein each of R₁, R₂, R₃, R₄, R₅, and R₇ is hydrogen, R₆, is methyl, and X is sulfur. The method provides for using compound 11 for treating cancer. In one aspect, cancer is not e colon cancer.

Some compounds of the invention were also tested in vivo in the B6BCL-2 transgenic mouse. Compounds 3, 5, and 11 of the invention, as shown above, exerted in vivo activities equal to, or better than, known compounds gossypol and apogossypol. Another known compound apogossypolone was not effective at all. Gossypol is described, e.g., in U.S. Pat. No. 7,186,708. Apogossypol is described, e.g., in Meyers A. I.; Willemsen J. J., Tetrahedron Letters, vol. 37, No. 6, February, 51996, pp. 791-792. The potency of the compounds in terms of in vivo efficacy was in the following order: compound 11>compound 5>compound 3=apogossypol=gossypol. In terms of toxicity, the severity of toxic effect diminished in the following order: gossypol>>>apogossypol>compound 5=compound 3=compound 11.

According to another embodiment, any above-described compound can be used for the manufacture of a medicament for the treatment of a pathological condition or symptom in a mammal, such as a human. The medicament can be directed to the treatment of cancer, within the limitations described above. A compound having the structure 11 may not to be preferred for treating colon cancer.

According to another embodiment, pharmaceutical compositions are provided, the pharmaceutical compositions comprising any above-described compound, or pharmaceutically acceptable salts, hydrates, or solvates thereof, and a pharmaceutically acceptable diluent or carrier. The pharmaceutical compositions can be used to treat cancer. The pharmaceutical compositions can further optionally include one or more additional therapeutic anti-cancer agents, including, but not limited to, such agents as (1) alkaloids, including, microtubule inhibitors (e.g., Vincristine, Vinblastine, and Vindesine, etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-16], and Teniposide [VM-26], etc.), and agents that target topoisomerase I (e.g., Camptothecin and Isirinotecan [CPT-11], etc.); (2) covalent DNA-binding agents [alkylating agents], including, nitrogen mustards (e.g., Mechlorethamine, Chlorambucil, Cyclophosphamide, Ifosphamide, and Busulfan [Myleran], etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); (3) noncovalent DNA-binding agents [antitumor antibiotics], including, nucleic acid inhibitors (e.g., Dactinomycin [Actinomycin D], etc.), anthracyclines (e.g., Daunorubicin [Daunomycin, and Cerubidine], Doxorubicin [Adriamycin], and Idarubicin [Idamycin], etc.), anthracenediones (e.g., anthracycline analogues, such as, [Mitoxantrone], etc.), bleomycins (Blenoxane), etc., and plicamycin (Mithramycin), etc.; (4) antimetabolites, including, antifolates (e.g., Methotrexate, Folex, and Mexate, etc.), purine antimetabolites (e.g., 6-Mercaptopurine [6-MP, Purinethol], 6-Thioguanine [6-TG], Azathioprine, Acyclovir, Ganciclovir, Chlorodeoxyadenosine, 2-Chlorodeoxyadenosine [CdA], and 2′-Deoxycoformycin [Pentostatin], etc.), pyrimidine antagonists (e.g., fluoropyrimidines [e.g., 5-fluorouracil (Adrucil), 5-fluorodeoxyuridine (FdUrd) (Floxuridine)] etc.), and cytosine arabinosides (e.g., Cytosar [ara-C] and Fludarabine, etc.); (5) enzymes, including, L-asparaginase, and hydroxyurea, etc.; (6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole [Arimidex], etc.); (7) platinum compounds (e.g., Cisplatin and Carboplatin, etc.); (8) monoclonal antibodies conjugated with anticancer drugs, toxins, and/or radionuclides, etc.; (9) biological response modifiers (e.g., interferons [e.g., IFN-.alpha., etc.] and interleukins [e.g., IL-2, etc.], etc.); (10) adoptive immunotherapy; (11) hematopoietic growth factors; (12) agents that induce tumor cell differentiation (e.g., all-trans-retinoic acid, etc.); (13) gene therapy agents; 14) antisense therapy agents; (15) tumor vaccines; (16) agents directed against tumor metastases (e.g., Batimistat, etc.), (17) inhibitors of angiogenesis, and (11) selective serotonin reuptake inhibitors (SSRI's).

Representative, but non-limiting examples of suitable SSRIs that may be used include sertraline (e.g., sertraline hydrochloride, marketed under the trademark “Zoloft®” by Pfizer, Inc.) or sertraline metabolite, fluvoxamine (e.g., fluvoxamine melate, marketed under the trademark “Luvox®” by Solvay Pharmaceuticals, Inc.), paroxetine (e.g., paroxetine hydrochloride, marketed under the trademark “Paxil®” by SmithKline Beecham Pharmaceuticals, Inc.), fluoxetine (e.g., fluoxetine hydrochloride, marketed under the trademarks “Prozac®” or “Sarafem®” by Eli Lilly and Company) and citalopram (e.g., citalopram hydrobromide, marketed under the trademark “Celexa®” by Forest Laboratories, Parke-Davis, Inc.), and metabolites thereof. Additional examples include venlafaxine (e.g., venlafaxine hydrochloride marketed under the trademark “Effexor®” by Wyeth-Ayerst Laboratories), mirtazapine (e.g., marketed under the trademark “Remeron®” by Organon, Inc.), buspirone (e.g., buspirone hydrochloride marketed under the trademark “Buspar®” by Bristol-Myers Squibb), trazodone (e.g., trazodone hydrochloride marketed under the trademark “Desyrel®” by Bristol-Myers Squibb and Apothecon), nefazadone (e.g., nefazodone hydrochloride marketed under the trademark “Serzon®” by Bristol-Myers Squibb), clomipramine: (e.g., clomipramine hydrochloride marketed under the trademark “Anafranil®” by Novopharm, LTD, Ciba, and Taro Pharmaceuticals), imipramine (e.g., imipramine hydrochloride marketed under the trademark “Tofranil®” by Glaxo-Welcome, Inc.), nortriptyline (e.g., Nortriptyline hydrochloride marketed under the trademark “Nortrinel®” by Lundbeck), mianserine (e.g., marketed under the trademark “Tolvon®” by Organon, Inc.), duloxetine (e.g., duloxetine hydrochloride marketed by Eli Lilly and Company), dapoxetine (e.g., dapoxetine hydrochloride marketed by ALZA Corporation), litoxetine (e.g., litoxetine hydrochloride marketed by Synthelabo Recherche (L.E.R.S.), Bagneux, France), femoxetine, lofepramine (e.g., marketed under the trademark “Gamonil®” by MERCK & Co., Inc.), tomoxetine (e.g., marketed by Eli Lilly and Company). The present invention encompasses SSRIs that are currently used, or those later discovered or formulated. SSRIs, including those listed above, may be administered orally in an amount between about 2 mg and about 2,500 mg daily.

In the broad sense, any cancer or tumor (e.g. hematologic and solid tumors) may be treated according to embodiments of the invention. Exemplary cancers that may be treated according to embodiments of the invention include, but are not limited to, head and neck cancer, brain cancer (e.g. glioblastoma multifoma) breast cancer, colorectal cancer, esophageal cancer, gastric cancer, hepatic cancer, bladder cancer, cervical cancer, endometial cancer, lung cancer (non-small cell), ovarian cancer and other gynecological cancers (e.g. tumors of the uterus and cervix), pancreatic cancer, prostate cancer, renal cancer, choriocarcinoma (lung cancer), skin cancer (e.g. melanoma, basal cell carcinoma), hairy cell leukemia, chronic lymphotic leukemia, acute lymphocytic leukemia (breast & bladder), acute myelogenous leukemia, meningeal leukemia, chronic myelogenous leukemia, and erythroleukemia. More commonly, the cancers treated include leukemia and B-cell cancers (e.g. lymphoma, multiple myeloma, and MDS.

The biological activity of compounds provided herein can be evaluated by in vitro and in vivo assays and procedures known in the art, including for example those described in Alley, M. C., et. al. Feasibility of Drug Screening with Panels of Human Tumor Cell Lines Using a Microculture Tetrazolium Assay. Cancer Research 48: 589-601, 1988; Grever, M. R., et. al. The National Cancer Institute: Cancer Drag Discovery and Development Program. Seminars in Oncology, Vol. 19, No. 6, pp 622-638, 1992, Boyd, M. R., and Paull, K. D. Some Practical Considerations and Applications of the National Cancer Institute In Vitro Anticancer Drug Discovery Screen. Drug Development Research 34: 91-109, 1995; Shoemaker, R. H. The NCI60 Human Tumour Cell line Anticancer Drug Screen. Nature Reviews, 6: 813-823, 2006, each of which is incorporated by reference in its entirety.

Non-limiting examples of autoimmune diseases that can be treated using compounds and methods of the present invention include rheumatoid arthritis, psoriatic arthritis, juvenile idiopathic arthritis, multiple sclerosis, systemic lupus erythematosus, myasthenia gravis, juvenile onset diabetes, glomerulonephritis, autoimmune thyroiditis, Behcet's disease, Crohn's disease, ulcerative colitis, bullous pemphigoid, sarcoidosis, psoriasis, ichthyosis, Graves ophthalmopathy, psoriasis, psoriasis inflammatory bowel disease, and asthma.

In cases where the compounds of the invention are sufficiently basic or acidic to form stable nontoxic acid or base salts, administration of the compounds as salts may be appropriate. Examples of pharmaceutically acceptable salts include organic acid addition salts formed with acids which form a physiological acceptable anion, for example, tosylate, methanesulfonate, acetate, citrate, malonate, tartarate, succinate, benzoate, ascorbate, ketoglutarate, and glycerophosphate. Suitable inorganic salts may also be formed, including hydrochloride, sulfate, nitrate, bicarbonate, and carbonate salts. Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Any tablets, troches, pills, capsules, and the like, which incorporate the inventive compounds, may also contain binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When there is a unit dosage form of the inventive compound in a capsule, it may contain, in addition to materials of the above type, a liquid carrier, such as a vegetable oil or a polyethylene glycol. Various other materials may be present as coatings or to otherwise modify the physical form of a solid unit dosage form. For instance) tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compounds of the present invention may also be administered intravenously or intraperitoneally by infusion or injection. Solutions of the compounds or salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations may contain a preservative to prevent the growth of microorganisms.

Sterile injectable solutions can be prepared by incorporating the compounds of the present invention in the sufficient therapeutic amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid. Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user, as known to those having ordinary skill in the art.

Embodiments of the present invention can be further illustrated by the following non-limiting examples.

### EXAMPLE 1

### Protein Expression and Purification

Recombinant full length BCL-X_{L} was produced from a pET-19b (Novagen) plasmid construct containing the entire nucleotide sequence for BID fused to an N-terminal poly-His tag. Unlabeled protein was expressed in E. coli BL21 in LB media at 37° C., with an induction period of 3-4 hours with 1 mM IPTG. ¹⁵N-labeled protein was similarly produced, with growth occurring in M9 media supplemented with 0.5 g/L ¹⁵NH₄Cl. Following cell lysis, soluble protein was purified over a Hi-Trap chelating column (Amersham, Pharmacia), followed by ion-exchange purification with a MonoQ (Amersham, Pharmacia) column. Final BID samples were dialyzed into a buffer appropriate for the subsequent experiments.

### EXAMPLE 2

### Molecular Modeling

Molecular modeling studies were conducted on several R12000 SGI Octane workstations with the software package Sybyl version 6.9 (TRIPOS). The docked structures of the compounds were initially obtained by Gold. Molecular models of compounds were energy-minimized with MAXIMN2 (Sybyl). For each molecule, 20 solutions were generated and ranked according to Goldscore. The solutions were finally ranked by visual inspection of the linked compounds in the deep hydrophobic groove on the surface of BCL-xL. Surface representations were generated by MOLCAD.

### EXAMPLE 3

### NMR Spectroscopy

For all NMR experiments, BCL-xL was exchanged into 50 mM phosphate buffer at pH 7.5 and measurements were performed at 30° C. 2D [¹⁵N,¹H]-HSQC spectra for BCL-xL were measured with 0.5 mM samples of ¹⁵N-labeled protein. All experiments were performed with a 600 MHz Bruker Avance spectrometer, both equipped with either a TXI probe or a TCI cryoprobe. In all experiments, dephasing of residual water signals was obtained with a WATERGATE sequence. In order to test the ability of test compounds to bind to Bcl-xL, a 25 μM sample of the protein was prepared and 1D ¹H NMR spectra were collected in absence and presence of test compounds. By observing the aliphatic region of the spectra, binding can be readily detected in these simple experiments due to chemical shift changes in active site methyl groups of Ile, Leu, Thr, Val or Ala (region between 0.8 and 0.3 ppm).

### EXAMPLE 4

### Synthetic Procedures

Rhodanine acetic acid or 3-methyl-2-(4-oxo-2-thioxothiazolidin-3-yl)butanoic acid, is added to a solution of the aldehyde (1:1.1 mmol ratio) in dimethylformamide (1 ml), and the mixture is stirred until it became homogenous. The mixture is then placed in the microwave (CEM), where it undergoes four cycles of 10-min heating (140° C., 1,000 W) and 5 min of cooling (25° C.). Water is then added to the solution, where precipitate is formed. The precipitate is collected via filtration, recrystallized from acetone/water, and dried to yield the desired compound.

### EXAMPLE 5

### Synthetic Procedures for Compounds of the Invention Comprising Naphthalene and Dihydronaphthalene Moieties

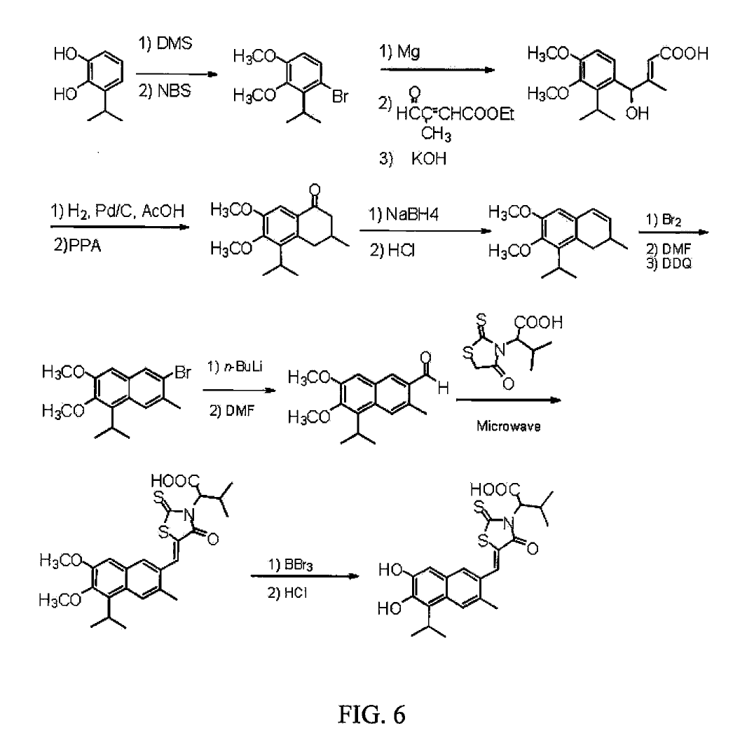

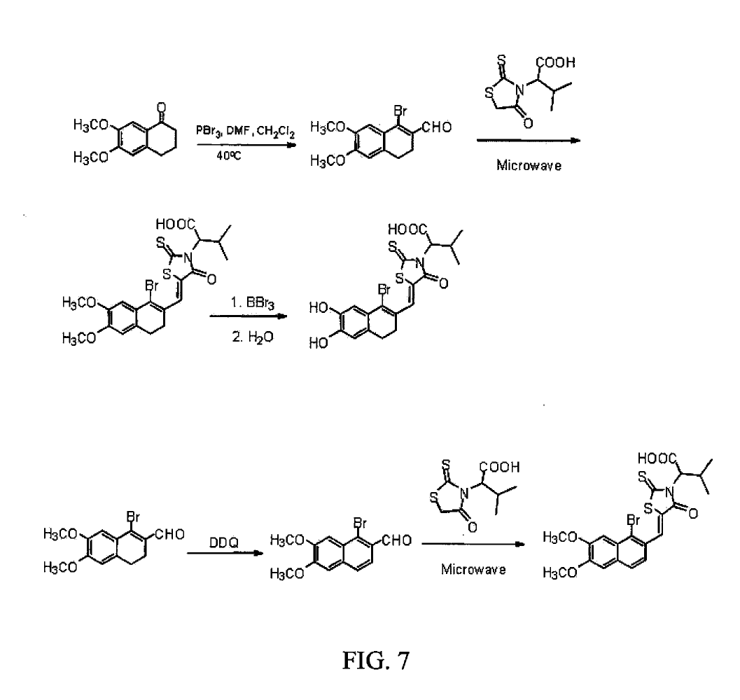

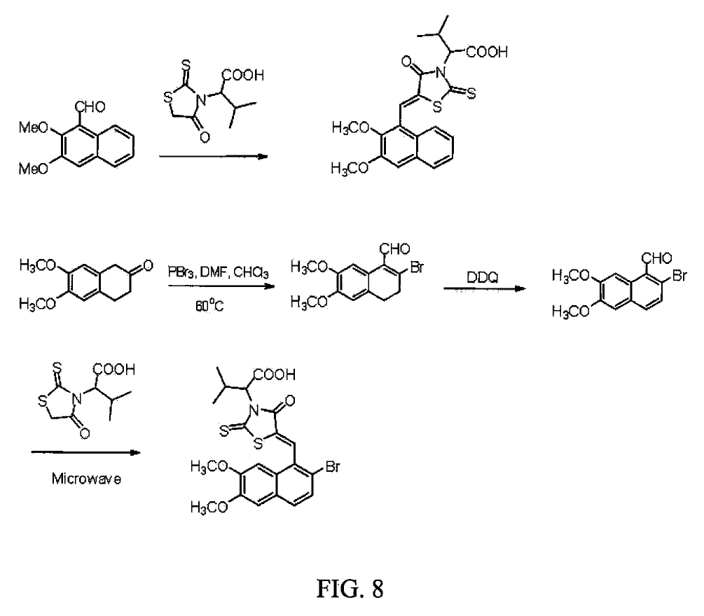

The reaction scheme shown on FIG. 6 demonstrates schematically one exemplary way of making some the above-described compounds. The reaction scheme shown on FIG. 7 demonstrates schematically one exemplary way of making some of the above-described compounds. The reaction scheme shown on FIG. 8 demonstrates schematically one exemplary way of making some of the above-described compounds.

### EXAMPLE 6

### Oral Delivery of Epalrestat In Vivo

Epalrestat (compound 11) was given to Triplet B6Bcl2 mice at a daily dose of 0.12 mmol/kg for 3 days (i.e., QDX3) through oral gavage. As a negative control, rhodanine acetic acid (which does not bind to Bcl-xL) was given at a daily dose of 0.12 mmol/kg for 3 days (i.e. QDX3) through oral gavage. Both Epalrestat and the negative control were dissolved in PBS.

After 3 days, the spleens of the animals were removed.

| | Spleen Weight |
|---|---|
| Epalrestat | 193.9 mg |
| Negative Control | 240.9 mg |

Hence, Epalrestat induced shrinkage of spleen whether dosed orally or intraperitoneally. Intraperitoneal injection induced more % shrinkage than oral dosing, however the results clearly indicate that Epalrestat can be administered in both ways, orally or intraperitoneally. Thus the compounds of the invention and Epalrestat in particular, can be administered in humans either orally or intravenously.

We should note that there was no weight loss or signs of toxicity via physical exam, indicating that Epalrestat is a safe drug, and qualitatively safer than ApoG given the lack of clear signs of toxicity via the intraperitoneal and oral routes of administration.

Although the invention has been described with reference to the above examples, it will be understood that modifications and variations are encompassed within the spirit and scope of the invention. Accordingly, the invention is limited only by the following claims.

What is claimed is:

1. A compound having the structure A, or a pharmaceutically acceptable salt, hydrate, N-oxide, or solvate thereof: 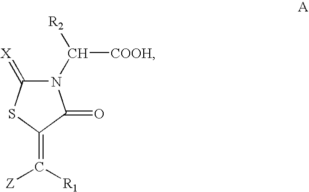
wherein:
each of R₁ and R₂ comprises hydrogen, a substituted or unsubstituted straight-chained aliphatic group, a halogen, an alkoxyl, a halogen-substituted alkyl, a halogen-substituted alkoxyl, hydroxyl, carboxyl, cyano group, an amido group, a substituted or unsubstituted cycloaliphatic group, a substituted or unsubstituted aromatic group, or a substituted or unsubstituted heterocyclic group;
X comprises oxygen, sulfur, or imino group; and
Z is a moiety selected from the group having the structures I, II, and III: 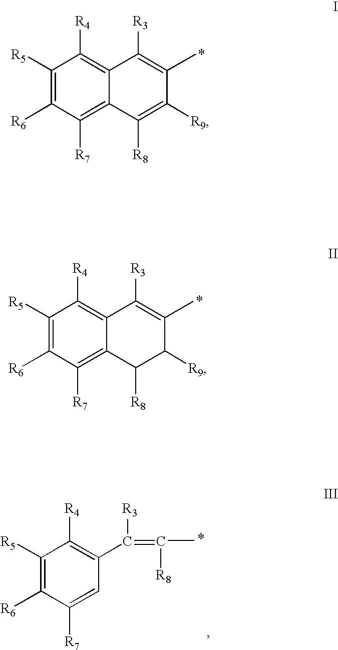
wherein:
each of R₃, R₄, R₅, R₆, R₇, R₉, and R₉ is hydrogen, a substituted or unsubstituted straight-chained aliphatic group, a halogen, an alkoxyl, a halogen-substituted alkyl, a halogen-substituted alkoxyl, hydroxyl, carboxyl, cyano group, an amido group, a, substituted or unsubstituted cycloaliphatic group, a substituted or unsubstituted aromatic group, or a substituted or unsubstituted heterocyclic group; and
symbol* indicates the point of attachment of the moiety Z of the immediately adjacent carbon in the C(═O)—R₁, structure.

2. The compound of claim 1, wherein Z is the moiety I.

3. The compound of claim 2, wherein each of R₁, R₂, R₃, R₄, R₅, R₆, R₇, R₈, and R₉ is selected from a group consisting of hydrogen, methyl, n-propyl, iso-propyl, fluorine, chlorine, bromine, phenyl, hydroxyl, methoxy, trifluoromethyl, trifluoromethoxy, cyano, carboxyl, and —C(O)NH₂.

4. The compound of claim 1, wherein Z is the moiety II.

5. The compound of claim 4, wherein each of R₁, R₂, R₃, R₄, R₅, R₆, R₇, R₈, and R₉ is selected from a group consisting of hydrogen, methyl, n-propyl, iso-propyl, fluorine, chlorine, bromine, phenyl, hydroxyl, methoxy, trifluoromethyl, trifluoromethoxy, cyano, carboxyl, and —C(O)NH₂.

6. The compound of claim 1, wherein Z is the moiety III.

7. The compound of claim 6, wherein each of R₁, R₂, R₃, R₄, R₅, R₆, R₇, R₈ and R₉ is selected from a group consisting of hydrogen, methyl, n-propyl, iso-propyl, fluorine, chlorine, bromine, phenyl, hydroxyl, methoxy, trifluoromethyl, trifluoromethoxy, cyano, carboxyl, and —C(O)NH₂.

8. The compound of claim 1, wherein the compound is 2-[5-(2-methyl-3-phenylallylidene)-4-oxo-2-thioxothiazolidin-3-yl]-2-phenylacetic acid.

9. The compound of claim 8, having the formula 1:

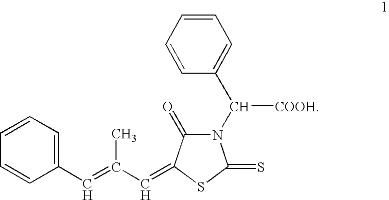

10. The compound of claim 1, wherein the compound is 3-methyl-2-[5-(2-methyl-3-phenylallylidene)-4-oxo-2-thioxothiazolidin-3-yl]butanoic acid.

11. The compound of claim 10, having the formula 2:

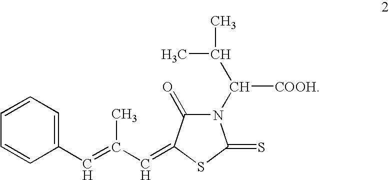

12. The compound of claim 1, selected from the group consisting of:
2-[5-(1-isopropyl-2,3-dimethoxy-7-methylnaphthalen-6-yl-methylene)-4-oxo-2-thioxothiazolidin-3-yl]-3-methylbutanoic acid;
2-[5-((2,3-dihydroxy-1-isopropyl-7-methylnaphthalen-6-yl)methylene)-4-oxo-2-thioxothiazolidin-3-yl]-3-methylbutanoic acid;
2-[5-((4-bromo-1,2-dihydro-6,7-dimethoxynaphthalen-3-yl)methylene)-4-oxo-2-thioxothiazolidin-3-yl]-3-methylbutanoic acid;
2-[5-((4-bromo-1,2-dihydro-6,7-dihydroxynaphthalen-3-yl)methylene)-4-oxo-2-thioxothiazolidin-3-yl]-3-methylbutanoic acid;
2-[5-((1,2-dimethoxynaphthalen-3-yl)methylene)-4-oxo-2-thioxothiazolidin-3-yl]-3-methylbutanoic acid;
2-[5-((naphthalen-2-yl)methylene)-4-oxo-2-thioxothiazolidin-3-yl]acetic acid;
3-methyl-2-[4-oxo-5-(3-phenylallylidene)-2-thioxothiazolidin-3-yl]butanoic acid; and
2-[4-oxo-5-(3-phenylallylidene)-2-thioxothiazolidin-3-yl]propanoic acid.

13. The compound of claim 12, having the formulae selected from the group 3-10:

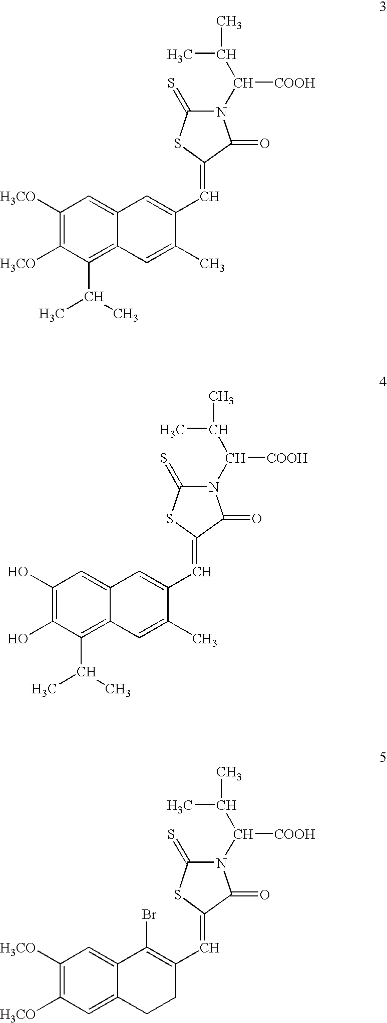

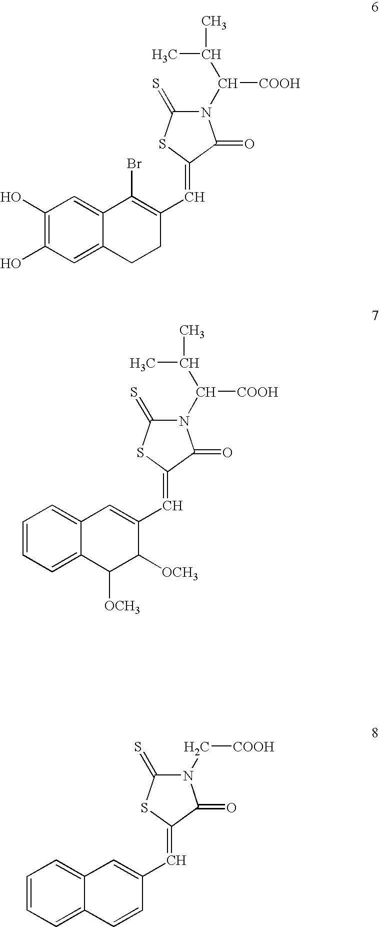

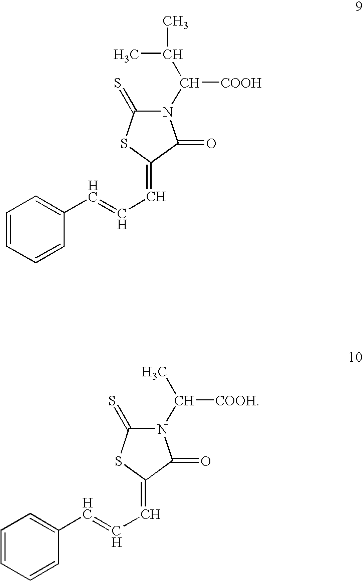

14. A method for treating a disease or a disorder, comprising administering to a subject in need thereof a therapeutically effective amount of at least one compound of claim 1, or a pharmaceutically acceptable salt, hydrate, N-oxide, or solvate thereof, thereby treating the disease or the disorder.

15. The method of claim 14, wherein the disease or the disorder is cancer.

16. The method of claim 14, wherein the treatment includes inhibition of activity of at least one BCL-2 family protein.

17. A method for treating a cancer, comprising administering to a subject in need thereof a therapeutically effective amount of compound having the structure 11, or a pharmaceutically acceptable salt, hydrate, N-oxide, or solvate thereof:

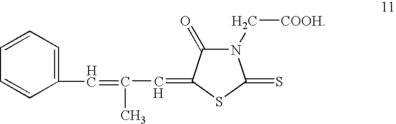

18. The method of claim 14 or 17, comprising administering the compound in combination with an anti-cancer agent.

19. A method of treating cancer or an autoimmune disease in a subject having at least one elevated BCL-2 family protein expression level comprising administering to the subject a therapeutically effective amount of compound having the structure 11, or a pharmaceutically acceptable salt, hydrate, N-oxide, or solvate thereof:

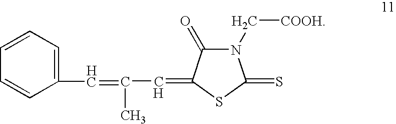

20. The method of claim 19, further comprising determining whether the subject is responsive to a therapy that utilizes the compound having the structure 11, or a pharmaceutically acceptable salt, hydrate, N-oxide, or solvate thereof, comprising determining the level of at least one of the BCL-2 family protein in the subject and comparing to a normal control sample, wherein an elevated level is indicative of a subject responsive to the therapy that utilizes compound having the structure 11, or a pharmaceutically acceptable salt, hydrate, N-oxide, or solvate thereof.

21. A method of determining whether a subject is responsive to a therapy that utilizes compound having the structure 11, or a pharmaceutically acceptable salt, hydrate, N-oxide, or solvate thereof: 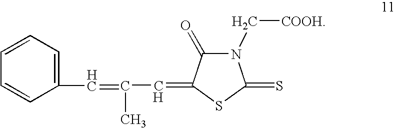
comprising determining the level of at least one of the BCL-2 family protein in the subject and comparing to a normal control sample, wherein an elevated level is indicative of a subject responsive to the therapy that utilizes compound having the structure 11, or a pharmaceutically acceptable salt hydrate, N-oxide, or solvate thereof.

22. The method of claim 20 or 21, wherein the determination is made based on a sample from the subject.

23. The method of claim 19, wherein the sample is a biological fluid or tumor sample.

24. The method of claim 19 or 21, wherein the BCL-2 family polynucleotide or polypeptide is selected from BCL-X_{L}, BCL-2, BCL-W, BCL-B, BFL-1, or MCL-1.

25. A method of inducing apoptosis in a cell having a level of at least one of the BCL-2 family protein member greater than levels in a control cell, comprising administering to the cell an effective amount of compound having the structure 11, or a pharmaceutically acceptable salt, hydrate, N-oxide, or solvate thereof: 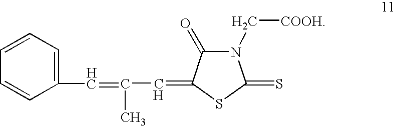
to reduce the level of Bcl-2 family protein(s) and induce apoptosis in the cell.

26. The method of claim 25, wherein the cell is a cancer cell.

27. The method of claim 25, wherein the cell is a cell of the immune system.

28. A method of determining the effectiveness of a therapeutic regimen including administration of compound having the structure 11, or a pharmaceutically acceptable salt, hydrate, N-oxide, or solvate thereof: 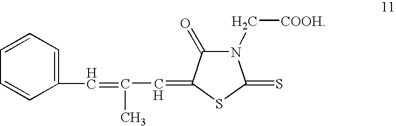
in a subject comprising comparing the level of a BCL-2 family protein in a cell of the subject prior to and during treatment with compound having the structure 11, or a pharmaceutically acceptable salt, hydrate, N-oxide, or solvate thereof, wherein a decreased level of BCL-2 family protein is indicative of effectiveness of the therapy that utilizes compound having the structure 11, or a pharmaceutically acceptable salt, hydrate, N-oxide, or solvate thereof.

29. The method of claim 28, wherein the subject has cancer.

30. The method of claim 28, wherein the subject has an autoimmune disorder.
